(19) United States
(12) Patent Application Publication
(10) Pub. No.: US 2005/0042171 A1
(43) Pub. Date: Feb. 24, 2005

(54) Pharmaceutical combinations comprising salmeterol and fluticasone proprionate for the treatment of asthma

(76) Inventors: Brian Charles Gavin, Dublin (IE); Jennifer Anne Sykes, Greenford (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/492,780

(22) PCT Filed: Oct. 10, 2002
(86) PCT No.: PCT/GB02/04602

(30) Foreign Application Priority Data
Oct. 12, 2001 (GB) 0124523.2

(51) Int. Cl. A61L 9/04; A61K 9/14

(52) U.S. Cl. 424/46; 514/171

(57) ABSTRACT

Methods for the prophylaxis or treatment of asthma by once daily administration of salmeterol and fluticasone propionate combinations as well as suitable pharmaceutical formulations are disclosed.

The present invention relates to the use of salmeterol and fluticasone propionate combinations for the once daily treatment of respiratory disorders, in particular asthma.

The combination of the beta-2 adrenergic agonist salmeterol or a physiologically acceptable salt thereof and the corticosteroid fluticasone propionate has been described in GB 2 235 627 for use in the treatment of asthma and other respiratory disorder via a twice daily (bis in diem—b.i.d) dosing regimen. The combination of salmeterol xinafoate and fluticasone propionate is now used clinically in the treatment of asthma. It is indicated for b.i.d. dosing.

Fluticasone propionate is an anti-inflammatory corticosteroid, described in GB 2088877, and is systematically named S-fluoromethyl-6α,9α-difluoro-11β-hydroxy-16α-methyl-17α-propionyloxy-3-oxoandrosta-1,4-diene-17β-carbothioate. Fluticasone propionate is now used clinically for the treatment of bronchial asthma and related disorders. Fluticasone propionate is indicated for b.i.d. dosing for the maintenance treatment of asthma.

GB 2 140 800 describes phenethanolamine compounds which are β₂-adrenoreceptor agonists including 4-hydroxy-α^{†}-[[[6-(4-phenylbutoxy)hexyl]-amino]methyl]-1,3-benzenedimethanol 1-hydroxy-2-naphthalenecarboxylate (salmeterol xinafoate) which is now used clinically in the treatment of bronchial asthma and related disorders. Salmeterol is now used clinically for the treatment of bronchial asthma and related disorders. It is indicated for b.i.d. dosing.

Asthma is a condition characterised by variable, reversible obstruction of the airways which is caused by a complex inflammatory process within the lungs. In most cases, this process is initiated and maintained by the inhalation of antigens by sensitive atopic individuals (extrinsic asthma). However, in some patients it is caused by other mechanisms which at present are poorly understood but do not involve an allergic process (intrinsic asthma). The disease has therefore two components, spasm of the bronchial (or breathing) tubes and inflammation or swelling of the breathing tubes.

Selective β₂-adrenoceptor agonists such as salbutamol have been used successfully and effectively by inhalation for the immediate relief of spasm in asthma. Salmeterol has a prolonged duration of action (“long acting”) of selective β₂-adrenoceptor antagonism enabling longer term control of bronchospasm and in reflection of this is included as a “controller medication” in international treatment guidelines such as GINA (Global Initiative For Asthma), (NHLBI/WHO Workshop Report, National Institutes of Health, National Heart Lung and Blood Institute, NIH Publication No. 95-3659, January 1995, and A Practical Guide for Public Health Care Professionals, National Institutes of Health, National Heart Lung and Blood Institute, NIH Publication No. 95-3659A, December 1995).

Anti-inflammatory corticosteroids such as, for example, fluticasone propionate have also been administered by inhalation in the treatment of asthma, although unlike β₂-adrenoceptor agonists the therapeutic benefits resulting from reduced inflammation may not be immediately apparent.

Fluticasone propionate, salmeterol xinafoate, and combinations of salmeterol xinafoate and fluticasone propionate, have previously only been proposed for the treatment or prophylaxis of asthma on the basis of a twice daily dose regimen. We have now surprisingly found that, in some patient populations, asthma can be satisfactorily controlled by the use of a combination of salmeterol or a physiologically acceptable salt thereof and fluticasone propionate on a once daily basis.

Accordingly, the present invention provides a method for prophylaxis or treatment of asthma in a mammal, such as a human, which comprises administering an effective amount of a combination of salmeterol or a physiologically acceptable salt thereof, such as the xinafoate salt, and fluticasone propionate on a once daily basis. In particular, there is provided a method for prophylaxis or treatment of mild or moderate asthma, especially persistent asthma, in a mammal, such as a human, which comprises administering an effective amount of a combination of salmeterol or a physiologically acceptable salt thereof, such as the xinafoate salt, and fluticasone propionate on a once daily basis.

In the alternative, there is provided the use of a combination of salmeterol or a physiologically acceptable salt thereof, such as the xinafoate salt, and fluticasone propionate for the manufacture of a medicament for the prophylaxis or treatment of asthma on a once daily basis. In particular, there is provided the use of a combination of salmeterol or a physiologically acceptable salt thereof, such as the xinafoate salt, and fluticasone propionate for the manufacture of a medicament for the prophylaxis or treatment of mild or moderate asthma, especially persistent asthma, on a once daily basis.

The severity of a patient's asthma can be classified as mild, moderate or severe depending on various criteria such as pulmonary function, symptamatology and the medication required in order to achieve effective control of the disease. Once daily dosing with a combination of salmeterol or a physiologically acceptable salt thereof, such as the xinafoate salt, and fluticasone propionate is particularly suitable for the treatment or prophylaxis of mild or moderate asthma, especially persistent asthma. Treatment may be initiated on the basis of once daily dosing, or may be stepped down from b.i.d. dosing to once daily dosing once a patient's asthma has stabilised. Once asthma stability for a patient has been achieved, it is desirable to titrate to the lowest effective dose to reduce the possibility of any potential side effects. Once daily dosing also allows greater flexibility to physicians in prescribing treatment for persistent asthma.

The need for a b.i.d. dosing regimen may discourage effective patient compliance. Once daily dosing offers a more convenient dosing regimen for patients and may lead to improved patient compliance with the dosing regimen. This can be especially important for paediatric patients.

As used herein, the term “treatment” means the improvement of clinical outcome, for example, alleviation of the symptoms of asthma, including nocturnal asthma, in particular prevention of bronchospasm, nocturnal cough, breathlessness and wheeze, and improvement in daytime lung function.

As used herein, the term “once daily” means that a patient's asthma is adequately controlled when the patient takes an effective dose of the combination of salmeterol or a physiologically acceptable salt thereof, such as the xinafoate salt, and fluticasone propionate once approximately every 24 hours. Preferably, a patient will take an effective dose of the combination at the same time in each 24 hour period, for example every morning, every afternoon or every evening, such that the individual doses are approximately 24 hours apart.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

It will be appreciated that the compounds of the salmeterol and fluticasone propionate combination may be administered simultaneously, either in the same or different pharmaceutical formulations, or sequentially. Where there is sequential administration, the delay in administering the second and any subsequent active ingredient should not be such as to lose the beneficial therapeutic effect of the combination of the active ingredients. In a preferred aspect of the invention, the salmeterol or its physiologically acceptable salt and the fluticasone propionate are administered as a combined pharmaceutical formulation. The weight/weight ratio of salmeterol to fluticasone administered according to the invention is preferably in the range 4:1 to 1:20.

The amount of salmeterol or a physiologically acceptable salt thereof, such as the xinafoate salt, and fluticasone propionate which is required to achieve a therapeutic effect will, of course, vary with the particular salt form, the route of administration, the subject under treatment, and the particular disorder or disease being treated. The combination of the invention may be administered to an adult human by inhalation at a dose of from 50 μg to 2000 μg per day, suitably 50 μg to 500 μg per day, more suitably 100 μg to 400 μg per day of fluticasone propionate and 50 μg to 200 μg per day, suitably 50 μg to 100 μg per day of salmeterol. The combination of the invention are preferably administered to an adult human by inhalation at a dose 50 μg of salmeterol, optionally in the form of the xinafoate salt, and 50 μg, 100 μg, 250 μg or 500 μg of fluticasone propionate per day, particularly preferably 50 μg of salmeterol, optionally in the form of the xinafoate salt, and 250 μg of fluticasone propionate per day.

The total daily dose may be inhaled in one actuation of an inhaler, for example a dry powder inhaler or a metered dose inhaler, or in more than one actuation, for example in 2, 3, or 4 actuations or “puffs”.

While it is possible for salmeterol or a physiologically acceptable salt thereof, such as the xinafoate salt, and fluticasone propionate to be administered as raw drugs, it is preferable to present each of them as a pharmaceutical formulation.

Thus according to a further aspect of the invention, there is provided a pharmaceutical formulation for the prophylaxis or treatment of asthma on a once daily basis comprising salmeterol or a physiologically acceptable salt thereof, such as the xinafoate salt, and fluticasone propionate, and a pharmaceutically acceptable carrier or excipient, and optionally one or more other therapeutic agents. Preferably, the pharmaceutical formulation is in a form which is suitable for administration by inhalation.

Hereinafter, the term “active ingredient” means salmeterol or a physiologically acceptable salt thereof, such as the xinafoate salt, and/or fluticasone propionate.

The formulations include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredients into association with the carrier which constitutes one or more accessory ingredients. In general the formulations are prepared by uniformly and intimately bringing into association the active ingredients with liquid carriers or finely divided solid carriers or both and then, if necessary, shaping the product into the desired formulation.

Formulations for inhalation include powder compositions which will preferably contain lactose, and spray compositions which may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluoroethane, carbon dioxide or other suitable gas. Suitable aerosol formulations include those described in EP 0372777 and WO93/11743. For suspension aerosols, the active ingredients should be micronised so as to permit inhalation of substantially all of the active ingredients into the lungs upon administration of the aerosol formulation, thus the active ingredients will have a particle size of less than 100 microns, desirably less than 20 microns, and preferably in the range 1 to 10 microns, for example, 1 to 5 microns.

Intranasal sprays may be formulated with aqueous or non-aqueous vehicles with the addition of agents such as thickening agents, buffer salts or acid or alkali to adjust the pH, isotonicity adjusting agents or anti-oxidants.

Capsules and cartridges or for example gelatin, or blisters of for example laminated aluminium foil, for use in an inhaler or insuflator may be formulated containing a powder mix of the active ingredients and a suitable powder base such as lactose or starch. In this aspect, the active ingredients are suitably micronised so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing a pharmaceutically effective dose, as hereinbefore recited, or an appropriate fraction thereof, of the active ingredient. Thus, in the case of formulations designed for delivery by metered dose pressurised aerosols, one actuation of the aerosol may deliver half of the therapeutically effective amount such that two actuations are necessary to deliver the therapeutically effective dose.

It should be understood that in addition to the ingredients particularly mentioned above, the formulations used according to the invention may include other agents conventional in the art having regard to the type of formulation in question, for example those suitable for oral administration may include flavouring agents. Furthermore, the claimed formulations include bioequivalents as defined by the US Food and Drugs Agency.

Furthermore, the combination of salmeterol or a physiologically acceptable salt hereof, such as the xinafoate salt, and fluticasone propionate used according to the present invention may be used in combination with or include a further active ingredient, for example anti-inflammatory agents (such as other corticosteroids (e.g. beclomethasone dipropionate, mometasone furoate, triamcinolone acetonide or budesonide) or NSAIDs (e.g. sodium cromoglycate, nedocromil sodium, PDE4 inhibitors, leukotriene antagonists, iNOS inhibitors, tryptase and elastase inhibitors, beta-2 integrin antagonists and adenosine 2a agonists)) or other β2-adrenoreceptor agonists (such as salbutamol, formoterol, fenoterol or terbutaline and salts thereof), anticholinergic agents (such as ipratropium, oxitropium or tiotropium) or antiinfective agents (e.g. antibiotics, antivirals).

For a better understanding of the invention, the following Examples are given by way of illustration.

### EXAMPLES

### Example 1

### 25/50 Salmeterol/Fluticasone Propionate Metered Dose Inhaler

| | Per actuation |
|---|---|
| Salmeterol Xinafoate | 36.3 microgram |
| Fluticasone Propionate | 50 microgram |
| 1,1,1,2-Tetrafluoroethane | to 75.0 mg |

The micronised active ingredients are weighed into an aluminium can, 1,1,1,2-tetrafluoroethane is then added from a vacuum flask and a metering valve is crimped into place.

Similar methods may be used for the formulation of Examples 2 to 4:

### Example 2

### 25/125 Salmeterol/Fluticasone Propionate Metered Dose Inhaler

| | Per actuation |
|---|---|
| Salmeterol Xinafoate | 36.3 microgram |
| Fluticasone Propionate | 125 microgram |
| 1,1,1,2-Tetrafluoroethane | to 75.0 mg |

### Example 3

### 25/250 Salmeterol/Fluticasone Propionate Metered Dose Inhaler

| | Per actuation |
|---|---|
| Salmeterol Xinafoate | 36.3 microgram |
| Fluticasone Propionate | 250 microgram |
| 1,1,1,2-Tetrafluoroethane | to 75.0 mg |

### Example 4

### 25/500 Salmeterol/Fluticasone Propionate Metered Dose Inhaler

| | Per actuation |
|---|---|
| Salmeterol Xinafoate | 36.3 microgram |
| Fluticasone Propionate | 500 microgram |
| 1,1,1,2-Tetrafluoroethane | to 75.0 mg |

### Example 5

### 50/50 Salmeterol/Fluticasone Propionate Dry Powder Inhaler

| | Per cartridge or blister |
|---|---|
| Salmeterol Xinafoate | 72.5 microgram |
| Fluticasone Propionate | 50 microgram |
| Lactose Ph.Eur. | to 12 mg |
| | or to 25 mg |

The active ingredients are micronised and bulk blended with the lactose in the proportions given above. The blend is filled into hard gelatin capsules or cartridges or in specifically constructed double foil blister packs (Rotadisks blister packs, Glaxo Group trade mark) to be administered by an inhaler such as the Rotahaler inhaler (Glaxo Group, trade mark) or in the case of the blister packs with the Diskhaler or Diskus inhalers (Glaxo Group trade marks).

Similar methods may be used for the formulation of Examples 6 to 8:

### Example 6

### 50/100 Salmeterol/Fluticasone Propionate Dry Powder Inhaler

| | Per cartridge or blister |
|---|---|
| Salmeterol Xinafoate | 72.5 microgram |
| Fluticasone Propionate | 100 microgram |
| Lactose Ph.Eur. | to 12 mg |
| | or to 25 mg |

### Example 7

### 50/100 Salmeterol/Fluticasone Propionate Dry Powder Inhaler

| | Per cartridge or blister |
|---|---|
| Salmeterol Xinafoate | 72.5 microgram |
| Fluticasone Propionate | 250 microgram |
| Lactose Ph.Eur. | to 12 mg |
| | or to 25 mg |

### Example 8

### 50/100 Salmeterol/Fluticasone Propionate Dry Powder Inhaler

| | Per cartridge or blister |
|---|---|
| Salmeterol Xinafoate | 72.5 microgram |
| Fluticasone Propionate | 500 microgram |
| Lactose Ph.Eur. | to 12 mg |
| | or to 25 mg |

The application of which this description and claims forms part may be used as a basis for priority in respect of any subsequent application. The claims of such subsequent application may be directed to any feature or combination of features described herein. They may take the form of product, composition, method, or use claims and may include, by way of example and without limitation, the following claims:

1. A method for prophylaxis or treatment of asthma in a mammal, which comprises administering an effective amount of a combination of salmeterol or a physiologically acceptable salt thereof and fluticasone propionate on a once daily basis.

2. A method according to claim 1 wherein the salmeterol or physiologically acceptable salt thereof and fluticasone propionate are administered as a combined pharmaceutical formulation.

3. A method according to claim 1 in which the salmeterol or physiologically acceptable salt thereof and fluticasone propionate are administered by inhalation.

4. A method according to claim 1 in which the salmeterol is administered as the xinafoate salt.

5-13. (Canceled).

14. A method according to claim 2 in which the salmeterol or physiologically acceptable salt thereof and fluticasone propionate are administered by inhalation.

15. A method according to claim 2 in which the salmeterol is administered as the xinafoate salt.

16. A method according to claim 3 in which the salmeterol is administered as the xinafoate salt.

17. A method according to claim 14 in which the salmeterol is administered as the xinafoate salt.

18. A method according to claim 1 in which the effective amount is from 50 μg to 2000 μg per day fluticasone propionate and 50 μg to 200 μg per day salmeterol.

19. A method according to claim 1 in which the effective amount is from 100 μg to 400 μg per day fluticasone propionate and 50 μg to 200 μg per day salmeterol.

20. A method according to claim 1 in which the effective amount is from 100 μg to 400 μg per day fluticasone propionate and 50 μg to 100 μg per day salmeterol.

21. A method according to claim 3 in which the daily dose is inhaled in one actuation of an inhaler.

22. A method according to claim 14 in which the daily dose is inhaled in one actuation of an inhaler.

23. A method for prophylaxis or treatment of asthma in a human which comprises administering an effective amount of a combination of salmeterol or a physiologically acceptable salt thereof and fluticasone propionate on a once daily basis, wherein the salmeterol or physiologically acceptable salt thereof and fluticasone propionate are administered by inhalation as a combined pharmaceutical formulation in which the effective amount is from 100 μg to 400 μg per day fluticasone propionate and 50 μg to 100 μg per day salmeterol.

24. A method according to claim 23 in which the daily dose is inhaled in one actuation of an inhaler.

25. A method according to claim 23 in which the effective amount is administered by inhalation from a powdered dose inhaler.

26. A method according to claim 23 in which the effective amount is administered by inhalation from a metered dose inhaler.

27. A method for prophylaxis or treatment of asthma in a human which comprises administering an effective amount of a combination of salmeterol or a physiologically acceptable salt thereof and fluticasone propionate on a once daily basis, wherein the salmeterol or physiologically acceptable salt thereof and fluticasone propionate are administered by inhalation as a combined pharmaceutical formulation and in which the daily dose is inhaled in one actuation of an inhaler.

28. A method according to claim 27 in which the effective amount is administered by inhalation from a powdered dose inhaler.

29. A method according to claim 27 in which the effective amount is administered by inhalation from a metered dose inhaler.
